(19) United States
(12) Patent Application Publication
(10) Pub. No.: US 2017/0245472 A1
(43) Pub. Date: Aug. 31, 2017

(54) ENVIRONMENTAL CONTROL FOR ENCLOSURES BASED ON TOUCH-LESS MONITORING OF ANIMALS

(71) Applicant: VitalMetric, LLC, Waunakee, WI (US)

(72) Inventors: Michael Rosing, Madison, WI (US); David Reid, Hazel Green, WI (US); James F. McCain, Houston, TX (US); Dennis Dynneson, Waunakee, WI (US)

(21) Appl. No.: 15/441,878

(22) Filed: Feb. 24, 2017

Related U.S. Application Data
(60) Provisional application No. 62/299,650, filed on Feb. 25, 2016.

(51) Int. Cl. A01K 29/00; A61B 5/08; A01K 7/00; A01K 11/00; A01K 1/00; A01K 1/12

(52) U.S. Cl. CPC A01K 29/005; A01K 1/0047; A01K 1/12; A01K 7/00; A01K 11/006; A61B 5/0816

(57) ABSTRACT

Embodiments of the invention provide a system and a method for controlling environmental conditions of an enclosure for animals. At least one radar device can be used to detect an indicator of a biological parameter for at least one of the animals. A value for the biological parameter can be determined based upon the detected indicator. Operation of environmental management equipment for the enclosure can be controlled based upon the determined value for the biological parameter.

### CROSS-REFERENCE TO RELATED APPLICATIONS

This application claims priority to U.S. Provisional Patent Application, Ser. No. 62/299,650, filed Feb. 25, 2016, entitled Environmental Control for Enclosures Based on Touch-less Monitoring of Animals, the entire contents of which is hereby incorporated by reference herein in its entirety.

### FIELD

The present inventions relate to the field of environmental control systems for enclosures or other spaces used by animals.

### BACKGROUND

In many applications, it may be useful to control environmental parameters of enclosures or other spaces that are used by animals. For example, in the context of dairy operations, it has been recognized that milk production may decrease when high temperatures in an enclosure cause dairy cows in the enclosure to experience heat stress. Accordingly, many conventional enclosures for dairy cows employ environmental management equipment to help regulate enclosure (and animal) temperatures. For example, to help reduce the incidents and effects of heat stress, conventional dairy enclosures can include fans to circulate air and misting equipment to spray water for cooling. Environmental control for enclosures is not limited to dairy enclosures, however, and can also be useful in other settings and for other animals, including humans.

### SUMMARY

Some embodiments of the invention provide a system for environmental control of an enclosure for animals. In one or more particular examples of embodiments, an environmental control for enclosures is provided based upon touch-less monitoring of animals. In this regard, at least one radar device can be configured to detect an indicator of a biological parameter for at least one of the animals. One or more controllers in communication with the radar device can be configured to determine a value for the biological parameter based upon the detected indicator and control operation of environmental management equipment of the enclosure based upon the determined value for the biological parameter.

Some embodiments of the invention provide a method for controlling environmental conditions of an enclosure for animals. An indicator of a biological parameter for at least one of the animals can be detected with at least one radar device. A value for the biological parameter can be determined based upon the detected indicator. Operation of environmental management equipment of the enclosure can be controlled based upon the determined value for the biological parameter.

Some embodiments of the invention provide a device for touch-less monitoring of a plurality of animals in an enclosure that includes environmental management equipment. The device can include at least one radar device configured to detect an indicator of a biological parameter for at least one of the animals. The device can further include a communication interface in communication with the environmental management equipment, and one or more controllers in communication with the radar device and the communication interface. The one or more controllers can be configured to determine a value for the biological parameter based upon the detected indicator, and to cause the communication interface to communicate control signals to the environmental management equipment based upon the determined value for the biological parameter.

These and other features and advantages of devices, systems, and methods according to this invention are described in, or are apparent from, the following detailed descriptions of various examples of embodiments.

### BRIEF DESCRIPTION OF DRAWINGS

Various examples of embodiments of the systems, devices, and methods according to this invention will be described in detail, with reference to the following figures, wherein:

FIG. 1 is top plan view of an enclosure with a system for environmental control according to one embodiment of the invention;

FIG. 2 is a schematic view of a monitoring device for use with the system of FIG. 1;

FIG. 3 is a flow diagram of the environmental control system according to one or more examples of embodiments;

FIG. 4 is a circuit diagram of the environmental control system according to one or more examples of embodiments; and

FIG. 5 is a logic diagram illustrating one example of operation of the environmental control system.

It should be understood that the drawings are not necessarily to scale. In certain instances, details that are not necessary to the understanding of the invention or render other details difficult to perceive may have been omitted. It should be understood, of course, that the invention is not necessarily limited to the particular embodiments illustrated herein.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise specified or limited, the phrases “at least one of A, B, and C,” “one or more of A, B, and C,” and the like, are meant to indicate A, or B, or C, or any combination of A, B, and/or C, including combinations with multiple instances of A, B, and/or C and combinations with individual instances of A, B, and/or C.

Also as used herein, unless otherwise specified or limited, electronic components specified as being in “communication” with each other can be in communication directly (e.g., wirelessly or via wired connections) or indirectly (e.g., via an intermediary communication device). Further, it will be understood that separate components configured to exchange information (e.g., electrical signals) with each other can be viewed as being in “communication” with each other even if the components are both included in a larger device.

Also as used herein, unless otherwise specified or limited, an “enclosure” can refer to an area in which animals are at least partly confined by man-made structures. For example, enclosures for livestock can include roofed or open-air buildings such as barns, milking parlors, and so on, as well as roofed or other pens.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Generally, embodiments of the invention disclosed herein can utilize touch-less monitoring of biological parameters as a basis for control of environmental management systems for animals. In some embodiments, for example, a control system can include a radar device configured to detect indicators of one or more biological parameters for one or more animals associated with a particular enclosure. The detected indicators can be processed to determine representative values for the relevant parameters. The representative values can then be used as a basis for control of environmental conditions for the enclosure. This can generally result in more efficient and/or effective environmental control with corresponding benefits according to the setting.

In some embodiments, multiple monitoring devices or radar devices can be used, operating independently or cooperatively. In some embodiments, multiple types of radar devices can be used, operating independently or cooperatively. In some embodiments, animal subjects of touch-less monitoring can be monitored individually. For example, biological parameters for individual animals can be determined separately, followed by statistical (or other) analysis of the individual data as appropriate. In some embodiments, animal subjects of touch-less monitoring can be monitored in groups. For example, biological parameters for groups of animals can be determined in aggregate, followed by statistical (or other) analysis as appropriate.

In some embodiments, the invention disclosed herein can be usefully employed in the context of dairy operations. For example, a dairy installation can include a roofed (or other) enclosure to house a herd of dairy cows, including for milking operations. The enclosure can include environmental management equipment including fans and misting systems, as well as shutters or shutter systems to help maintain appropriate temperatures for the herd. In some embodiments, a control system can include a radar device configured for touch-less monitoring of one or more biological parameters, such as respiration rate, for cows within the enclosure. Based on data from the monitoring, a controller can determine whether the herd (or a portion thereof) is experiencing or is in danger of experiencing heat stress or other relevant conditions. The environmental management equipment can then be controlled accordingly. As one example, if an average respiration rate for the herd, as determined via touch-less monitoring, indicates the relatively widespread existence of heat stress, the environmental management equipment can be controlled to increase animal cooling (or otherwise regulate animal temperature), including through increased fan operation and/or increased misting.

Certain examples discussed herein address enclosures for dairy operations and, accordingly, monitoring of dairy cows to determine relevant biological parameters. It will be understood that embodiments the disclosed invention can alternatively (or additionally) be used in other agricultural settings, including in other milking operations (e.g., for goats). Further, it will be understood that embodiments of the disclosed invention can alternatively (or additionally) be used in non-agricultural settings, including for monitoring and regulation of human-centered activities, such as may occur in enclosed assembly lines, steel mills, and other industrial settings.

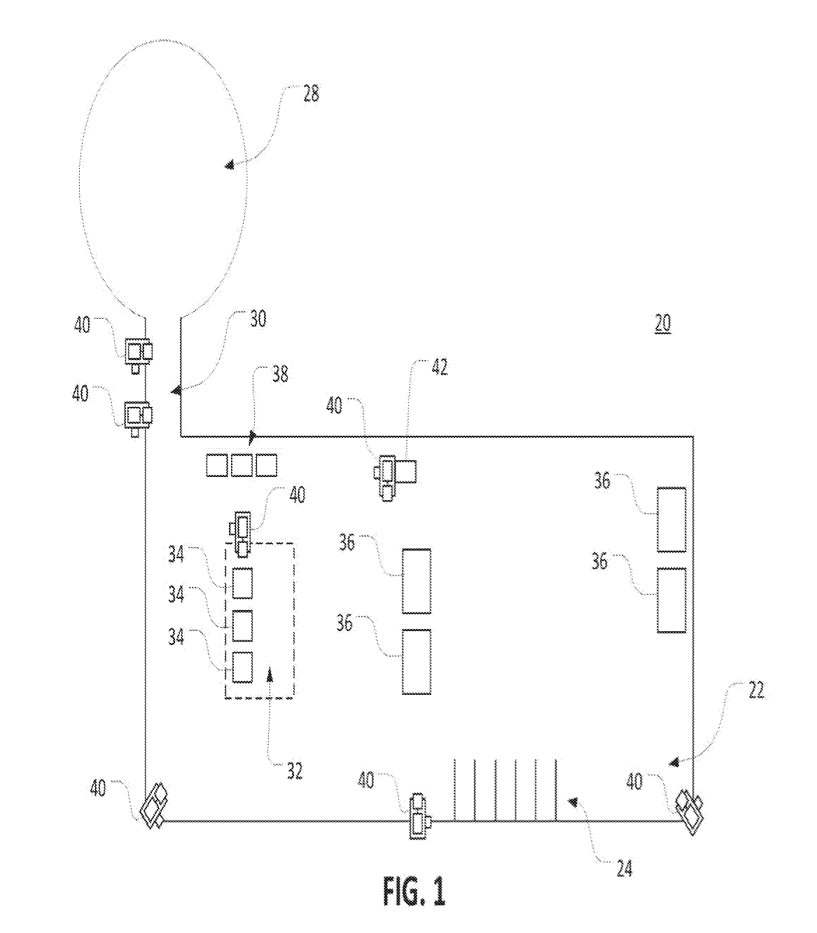

FIG. 1 illustrates an example enclosure 20, for the environmental control of which some embodiments of the invention can be deployed. In the embodiment illustrated, the enclosure 20 is an enclosure for dairy cows, including a pen area 22, a milking parlor 28, and an alleyway 30 extending between the pen area 22 and the milking parlor 28. Generally, the alleyway 30 can include a somewhat restricted area compared to the pen area 22, such that only a limited number of cows can pass through the alleyway 30 at a particular time. The enclosure 20 also includes a watering area 32 with a number of watering troughs 34. It will be understood that the configuration of the enclosure 20 is provided as an example only, and that embodiments of the disclosed invention can be used in various other enclosures.

The enclosure 20 can include various types of environmental management equipment configured to provide cooling or other environmental adjustments for the enclosure 20. In the embodiment illustrated, the environmental management equipment installed in the enclosure 20 includes a number of fans 36 as well as a misting array 38 configured to mist water onto nearby animals. In some embodiments, one or more of the fans 36 can be configured to move (e.g., pivot) in order to distribute the air flow from the fans 36 over different areas of the enclosure 20. In some embodiments, one or more of the fans 36 can include a fluid system (not shown), as may be useful, for example, to inject water droplets into the air flow from the fans 36. In other embodiments, one or more shutters may be provided, which may be coupled to a motor or other device operable to open and close the shutter over a window, door, or other opening. It will be understood that the location, number, and other aspects of the configuration of the fans 36 and the misting array 38 in the enclosure 20 is provided as an example only. In other embodiments, other types and configurations of fans, misters, and other environmental management equipment may be possible. For example, some enclosures 20 in colder climates can also include heating equipment.

As also noted above, embodiments of the disclosed invention can be used in contexts other than the enclosure 20. Accordingly, it will be understood that certain enclosure can include different (or additional) environmental management equipment than the enclosure 20. For example, some enclosures can include HVAC equipment capable of providing cooled, heated or otherwise conditioned air, liquid-circulation heating or cooling equipment, adjustable vents or covers for regulating natural or other air flow (e.g., adjustable window or door covers), and so on.

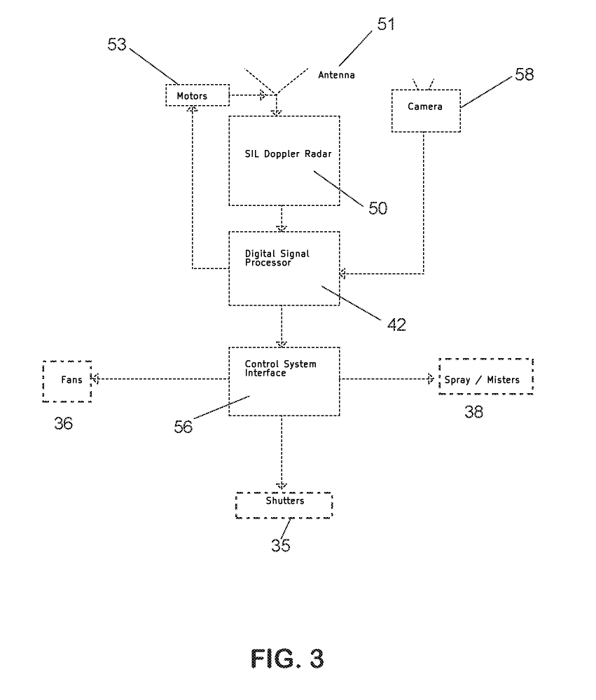

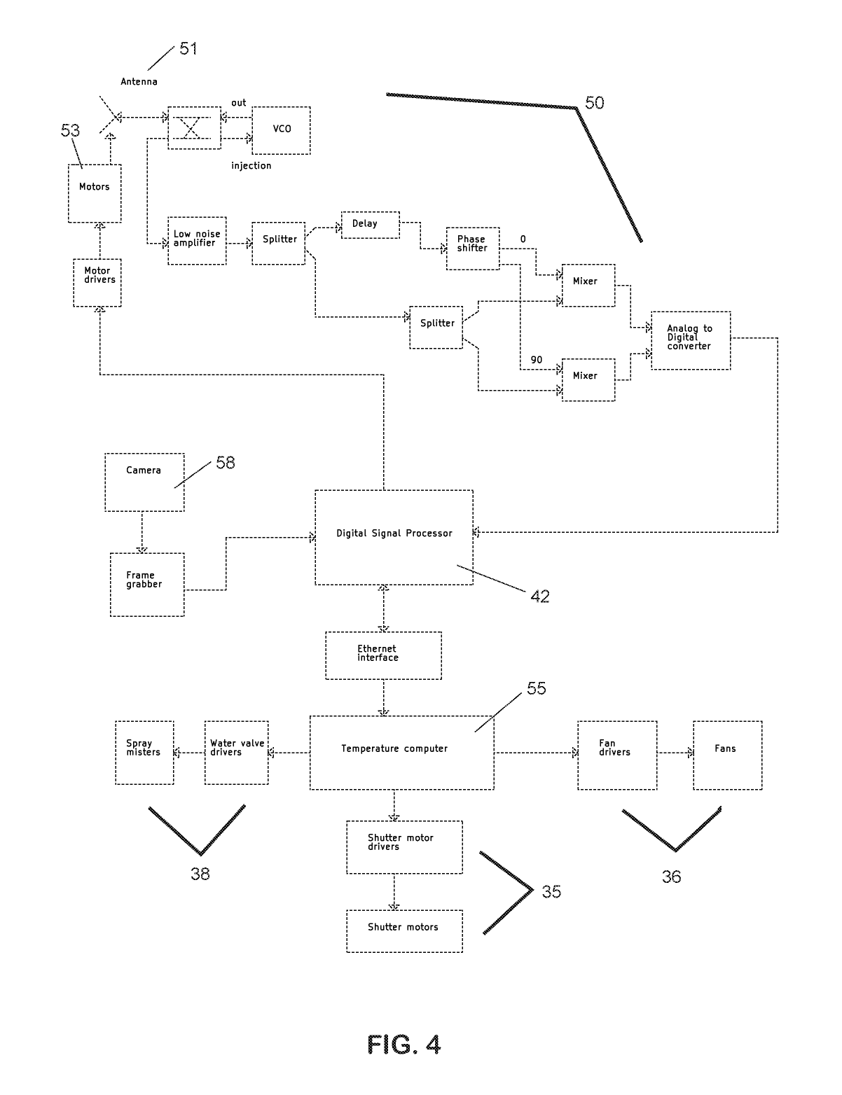

Referring to FIGS. 3-4, the environmental control system according to one or more examples of embodiments will be described. The environmental control system generally includes one or more of the above-described environmental management devices in communication with a controller or control system interface and a monitoring device. More specifically, as can be seen in FIGS. 3-4, one or more monitoring devices, which monitoring device may be a radar device 50, is in communication with a digital signal processor 42. The monitoring device 50 may have an antenna 51 coupled to a motor 53 for movement or rotation of the antenna 51. One or more cameras 58 may also be in communication with the digital signal processor 42. The digital signal processor 42 is configured to receive one or more signals communicating data from the monitoring device(s) or radar(s) 50 and/or the camera(s) 58 and process that data. The digital signal processor 42 is also in communication with the motor(s) 53 joined to the radar antenna 51. In one or more examples of embodiments, each radar system may be provided with its own digital processor. In alternative examples of embodiments, one digital signal processor may be in communication with a plurality of radar systems. The digital signal processor or each digital signal processor 42 is further in communication with one or more control system interface(s) 55 for the environmental management devices. The control system interface 55 is in communication with one or more spray or misting devices 38, one or more shutters 35, and/or one or more fans 36. The control system interface 55 is configured to receive data from the digital signal processor 42 and control the operation of one or more of the environmental management devices 35, 36, 38. While specific examples of devices are provided herein for illustration, one of skill in the art would understand that additional and/or alternative devices may be included in the environmental control system which devices accomplish the purposes provided.

In addition, while a single “control system interface” is described, there may be several control systems. For example, one control system may be or include in association with the radar 50—i.e., a control of where the antenna 51 points. Other examples include the controls for the fan system 36, spray system 38 and shutter system 35. Each of said devices may have its own set of controls or control system. As one illustrative example, there may be a plurality of fans in an enclosure. Each fan can run at several different speeds. There may be times when different fan speeds are required for different devices, and other times when each device may be operated at the same speed.

In accordance with some embodiments of the invention, one or more monitoring devices 40 can be deployed in the enclosure 20 in order to monitor biological parameters (e.g., respiration and/or heart rate or pulse) for the cows within the enclosure 20. The monitoring devices 40 can be deployed at various locations in the enclosure 20, including in the pen area 22, in the alleyway 30, near the watering area 32 (e.g., at each of the watering troughs 34), or elsewhere. In some embodiments, sets of the monitoring devices 40 can be deployed in related arrays. For example, an array of the monitoring devices 40 within (or directed towards) the alleyway 30 can include multiple monitoring devices 40 spaced (or directed) at predetermined (e.g., regular) intervals along the alleyway 30. However, one of skill in the art would understand that predetermined or regular intervals are not required.

In some embodiments, one or more of the monitoring devices 40 can be deployed at ground level. In some embodiments, one or more of the monitoring devices 40 can be deployed at elevated positions (including, but not limited to, at or near the internal roof of the enclosure 20) with monitoring equipment of the monitoring devices 40 accordingly directed at least partly downwardly, in order to monitor cows on the floor of the enclosure 20.

Generally, each of the monitoring devices 40 includes a sensor for detecting indicators for one or more biological parameters for the cows in the enclosure 20, an electronic controller (e.g., programmable circuit, general purpose computer, or other computing device), and a communication interface for communicating the detected indicators or other information relating thereto. In some embodiments, each of the monitoring devices 40 can be configured as an integral unit that can be moved and installed as a whole. In some embodiments, one or more of the monitoring devices 40 can be configured as a system of modules that can be moved and installed separately.

In some embodiments, a main processing unit 42 (e.g., a centralized electronic controller) can be in communication with one or more of the monitoring devices 40 and with the environmental management equipment (or a controller thereof) in order to control the environmental management equipment based upon information received from the monitoring devices 40. In some embodiments, one or more of the monitoring devices 40 can be in direct communication with the environmental management equipment in order to control the environmental management equipment based upon information detected or derived by the one or more monitoring devices 40. In some embodiments, the main processing unit 42 can be included in a particular one of the monitoring devices 40, with other monitoring devices transmitting information to the monitoring device 40 that includes the main processing unit 42, and with that monitoring device 40 controlling the environmental management equipment accordingly.

As indicated, in one or more examples of embodiments the system and/or method may be implemented by a processing unit, a microcontroller, or a computer system, or in combination with a computer system. The computer system may be or include a processor. The computers may be electronic devices for use with the methods and various components described herein and may be programmable computers which may be special purpose computers or general purpose computers that execute the system according to the relevant instructions. The computer system or portable electronic device can be an embedded system, a personal computer, notebook computer, server computer, mainframe, networked computer, workstation, handheld computer, as well as now known or future developed mobile devices, such as for example, a personal digital assistant, cell phone, smartphone, tablet computer, and the like. Other computer system configurations are also contemplated for use with the communication system including, but not limited to, multiprocessor systems, microprocessor-based or programmable electronics, network personal computers, minicomputers, smart watches, and the like. Preferably, the computing system chosen includes a processor suitable in size to efficiently operate one or more of the various systems or functions or attributes of the system described.

The system or portions thereof may also be linked to a distributed computing environment, where tasks are performed by remote processing devices that are linked through a communication network(s). To this end, the system may be configured or linked to multiple computers in a network including, but not limited to, a local area network, wide area network, wireless network, and the Internet. Therefore, information, content, and data may be transferred within the network or system by wireless means, by hardwire connection, or combinations thereof. Accordingly, the servers described herein communicate according to now known or future developed pathways including, but not limited to, wired, wireless, and fiber-optic channels.

Data, for example, sensor data or recommendations, may be sent or submitted via the Internet, wireless, and fiber-optic communication network(s), or created or stored on a particular device. In one or more examples of embodiments, data may be stored remotely or may be stored locally on the user's device or controller. In one example, data may be stored locally in files. Data may be stored and transmitted by and within the system in any suitable form. Any source code or other language suitable for accomplishing the desired functions described herein may be acceptable for use.

Furthermore, the computer or computers or portable electronic devices may be operatively or functionally connected to one or more mass storage devices, such as but not limited to, a database. The memory storage can be volatile or non-volatile, and can include removable storage media. Cloud-based storage may also be acceptable. The system may also include computer-readable media, which may include any computer-readable media or medium that may be used to carry or store desired program code that may be accessed by a computer. The invention can also be embodied as computer-readable code on a computer-readable medium. To this end, the computer-readable medium may be any data storage device that can store data which can be thereafter read by a computer system. Examples of computer-readable medium include read-only memory, random-access memory, CD-ROM, CD-R, CD-RW, magnetic tapes, flash drives, as well as other optical data storage devices. The computer-readable medium can also be distributed over a network-coupled computer system so that the computer-readable code is stored and executed in a distributed fashion.

A display (not shown) may be provided for display of data. To this end, a screen may be provided as part of the system. The screen may be a tablet or mobile computing device. The screen may be positioned where a user may observe the screen data.

Additionally, information may be stored in the system such that data can be used to provide feedback. In various embodiments, data may be stored in the screen, computing device, mobile device, or other suitable location.

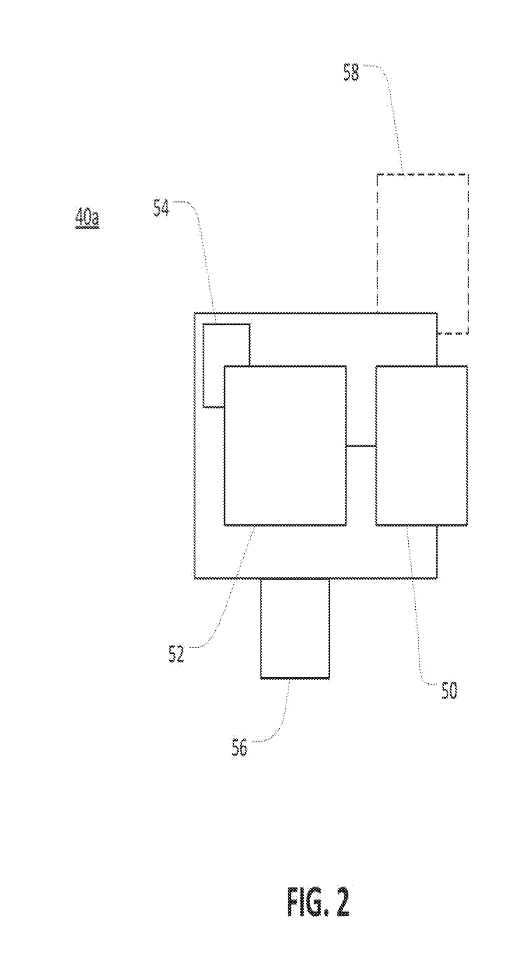

FIG. 2 illustrates a configuration of a monitoring device 40a, according to some embodiments of the invention. In the embodiment illustrated, the monitoring device 40a includes a radar device 50, an electronic controller 52, and a communication interface 54. In some example of embodiments, the monitoring device 40a may also include a processor. The radar device 50 can be generally configured to detect indicators relating to biological parameters such as respiration rates or heart rates or pulses for cows and/or other animals in the enclosure 20 and relay the indicators to the electronic controller 52 for processing. The electronic controller 52 can process the indicators in various ways, including, for example, processing the indicators to extract more useful information regarding the relevant biological parameters (e.g., actual respiration rates rather than raw or partially-processed signal data), and formatting the indicators or related information for transmission by the communication interface 54 to a remotely disposed device (e.g., the main processing unit 42).

In some embodiments, including as discussed in detail below, the electronic controller 52 can provide local control for a variety of operations for the monitoring device 40a. In some embodiments, the electronic controller 52 can be supplemented (or replaced) by a remotely located electronic controller, such as the main processing unit 42 (see FIG. 1), which can control one or more operations of the monitoring device 40a via the communication interface 54.

The communication interface 54 can be configured in various ways, depending on the needs of the monitoring device 40a and/or other aspects of the installation setting. In some embodiments, the communication interface 54 can include an antenna for wireless reception and transmission of information. In some embodiments, the communication interface 54 can include a wired (e.g., USB or co-axial cable) interface for non-wireless reception and transmission of information.

In some embodiments, the monitoring device 40a can also include a motive device 56. Generally, the motive device 56 can be configured to move part or all of the monitoring device 40a in a predetermined or otherwise controllable way. For example, in some embodiments, the motive device 56 can be configured to rotate the radar device 50, or the monitoring device 40a as a whole, through a predetermined angular scope, such that the radar device 50 can detect indicators of relevant biological parameters over a distributed area. In some embodiments, the motive device 56 can be configured to provide one-dimensional (“1-D”) movement, such as movement along a 1-D arc. This may be useful, for example, where the monitoring device 40a is disposed at ground level. In some embodiments, the motive device 56 can be configured to provide two-dimensional (“2-D”) movement, such as pivotal movement within a predetermined spherical sector. This may be useful, for example, where the monitoring device 40a is disposed overhead relative to target areas within the enclosure 20 (e.g., mounted at the roof of the enclosure 20). Additional dimensional movement is also contemplated.

In some embodiments, the monitoring device 40a can additionally (or alternatively) include or be configured to interoperate with other monitoring equipment. For example, in some embodiments, the monitoring device 40a can include (or be configured to operate in conjunction with) an imaging device 58, such as but not limited to a camera. As also discussed below, for example, the imaging device 58 can be configured as a visual, infrared, or other camera with an overlapping field of view with the radar device 50. In one example, a camera may be used to locate an animal, such as a cow, standing still and positioned sideways relative to the monitoring device, and then focus on and take the respiration reading from that animal. In other words, the antenna may rotate to locate and focus on a random cow that would have 2 attributes standing still, and standing sideways to the antenna.

As also noted above, the radar device 50 generally can be configured to detect indicators of one or more biological parameters. In some embodiments, the radar device 50 can be configured to detect movements of a cow (or other animal) that correspond to respiration rate. In some embodiments, the radar device 50 can be configured to itself derive a respiration rate (or other biological parameter) from such indicators. In some embodiments, the radar device 50 can provide data from detected indicators to a separate processing device (e.g., the electronic controller 52 or the main processing unit 42), and the separate processing device can derive the respiration rate (or other biological parameter) from the indicators.

In different embodiments, the radar device 50 can take a variety of different forms and can employ a variety of different technologies. In some embodiments, for example, the radar device can be configured to use one or more of pulsed Doppler radar, continuous wave Doppler radar, ultra-wideband radar, self-injection-locked (SIL) radar, single-antenna radar, and the like, and combinations of the foregoing. In some embodiments, the radar device 50 can employ non-traditional radar technologies, such as light-based radar (e.g., LED radar or “Leddar”, which can use 1-D or 2D time-of-flight analysis to locate and map objects).

In some embodiments, the radar device 50 can be configured to monitor a fixed area (e.g., a field of view of part of the enclosure 20 with a fixed angular range). In some embodiments, the radar device 50 can monitor a range of areas, including, for example, via sweeping through a predetermined 1-D or 2-D angular range. Various technologies can be employed to allow the radar device 50 to sweep through a range, including servo motors or other mechanical-motion technologies (e.g., as provided by the motive device 56), phased array technologies, meta-materials, and so on. In some embodiments, including as discussed below, multiple instances of the radar device 50 can be used in combination to monitor different (e.g., exclusive or overlapping) areas, including via fixed-area or swept-area monitoring.

As also noted above, the monitoring devices 40 (including the monitoring device 40a) can be deployed at different areas and in different relative configurations within the enclosure 20. In some embodiments, the monitoring devices 40 can be deployed in locations near which animals are likely to pass or congregate, such that relatively accurate measurements of particular indicators can be made for relatively large numbers of animals. Referring again to FIG. 1, for example, one or more of the monitoring devices 40 can be deployed along the alleyway 30, such that the monitoring devices 40 can detect indicators of biological parameters for cows passing through the alleyway 30. In some embodiments, this can include deployment of monitoring devices 40 with a predetermined (e.g., regular) spacing along the alleyway 30. Placement of the monitoring devices 40 along the alleyway 30 may be useful, for example, because the restricted area of the alleyway 30 can help to ensure that a particular monitoring device 40 is focused on detecting indicators for only one particular animal at a time, which can increase the accuracy and utility of measurements by the monitoring device 40. Similarly, in some embodiments, one or more monitoring devices 40 can be deployed at other locations, including nearby the watering troughs 34, nearby one or more pens 24, and so on.

In some embodiments, it may be useful to arrange the monitoring devices 40 in arrays relative to each other. For example, in some embodiments, two monitoring devices 40 can be deployed on opposite sides of a shared target area. This can be useful, for example, in helping to eliminate unwanted artifacts (e.g., from large-scale animal movement) from relevant monitored indicators. As another example, multiple monitoring devices 40 can be deployed to cover respective overlapping or non-overlapping regions, such that the monitoring devices 40 can collectively monitor relatively large areas of the enclosure 20. This may be useful, for example, to allow the monitoring devices 40 to relatively quickly and efficiently aggregate information regarding biological parameters for statistically significant portions of the herd.

In some embodiments, aspects of deployment for the monitoring devices 40 can depend on the type of radar device (e.g., the type of the radar device 50) employed by the monitoring devices 40. For example, where the radar device 50 (or the monitoring device 40a, generally) is configured to scan over a 1-D or 2-D area, it may be possible for the monitoring device 40a to replace multiple other monitoring devices 40. For example, it may be possible to replace an array of multiple monitoring devices 40 along the alleyway 30 with a smaller number of monitoring devices 40 (e.g., the single monitoring device 40a) that can scan over a 1-D or 2-D angular range that covers an appropriate portion (e.g., all or most) of the alleyway 30. Likewise, it may be possible to replace an array of multiple monitoring devices 40 at the watering area 32 (e.g., one monitoring device 40 per watering trough 34) with a smaller number of monitoring devices 40 that can scan over a 1-D or 2-D angular range that appropriately covers the watering area 32.

In some embodiments, it may be useful to utilize different radar frequencies at different monitoring devices 40 (or at different radar devices included in a single monitoring device 40). This can be useful for example, to help to prevent various monitoring devices 40 in an array (or various radar devices in a single monitoring device 40) from interfering with each other. For example, where a first and a second monitoring device 40 are deployed with overlapping scanning ranges (e.g., overlapping 1-D arc segments), it may be useful for the first monitoring device 40 to utilize a first radar frequency and for the second monitoring device 40 to utilize a second radar frequency that is different from the first radar frequency.

In some embodiments, the monitoring devices 40 may be relatively portable. As such, for example, the monitoring devices 40 can be fixed in particular locations as desired (e.g., via semi-permanent attachment to support structures of the enclosure 20), but then may be re-located as appropriate. In some embodiments, for example, monitoring of system performance (e.g., via the main processing unit 42) may indicate that a first set of monitoring devices 40 at particular locations (or orientations) tend to provide relatively useful data, while a second set of monitoring devices 40 at other locations (or orientations) tend to provide less useful data. The main processing unit 42 can accordingly recommend (e.g., via a user interface (not shown)) that the second set of monitoring devices 40 be relocated (or reoriented).

In some embodiments, radar monitoring via the monitoring devices 40 (e.g., via the radar device 50 of the monitoring device 40a) can proceed in combination with monitoring via other technologies. As also noted above, for example, the monitoring device 40a can include the imaging device 58 (see FIG. 2) (e.g., a camera), which can be configured as a visual, infrared, or other camera with an overlapping field of view with the radar device 50. In some embodiments, the imaging device 58 can be used to identify when an animal is relevantly within the field of view of the radar device 50, so that the radar device 50 can be used with relatively high confidence to gather relevant indicators of biological parameters. In some embodiments, the imaging device 58 can be used to identify when an animal within the field of view of the imaging device 58 or the radar device 50 is engaged in particular behavior (e.g., sitting, standing, panting, and so on), such that appropriate action may be taken. For example, upon detection by the imaging device 58 of an animal engaged in panting, the radar device 50 can be instructed to detect indicators for that animal, data indicating a likelihood of heat stress can be associated with other data recorded by the monitoring device 40a (e.g., indicators of respiration rate), or an alert can be provided (e.g., to the main processing unit 42) to prompt further action. In some embodiments, this can be usefully employed to calibrate the monitoring devices 40 (and the system in general) to better identify indicators of heat stress in the herd. While a camera 58 may be used to optically detect the presence of an animal, the camera may also or alternatively detect temperature. For example, infra-red or near infra-red detection may be used to detect the presence and location of an animal, as well as an animal's external temperature. An infra-red imaging device may also monitor ground and/or building temperatures. In one example, one or more imaging devices 58 are used to detect both optical and infra-red, or alternatively a hyperspectral imaging system may be used to provide high information density.

The monitoring devices 40, individually or collectively, can be configured to detect indicators relating to a variety of different biological parameters. In some embodiments, as also discussed above, the monitoring devices 40 can be configured to detect indicators relating to animal respiration. For example, with regard to the monitoring device 40a, the radar device 50 can be configured to detect minute animal movements corresponding to animal respiration rate, or other biological factors such as heart rate. Monitoring of other indicators may also (or alternatively) be possible, including indicators relating to larger scale movements, such as panting, herd behavior, or other indicators of animal stress.

In some embodiments, the monitoring devices 40 can internally process detected indicators in order to determine relevant biological parameters. For example, once indicators of movements relating to respiration are detected by the radar device 50, the electronic controller 52 (or the radar device 50 itself) can process the indicators to determine the respiration rate of the monitored animal.

In some embodiments, the monitoring devices 40 can transmit detected indicators to external systems for identification of relevant biological parameters. For example, once indicators of movements relating to respiration are detected by the radar device 50, data representing those indicators can be transmitted to the main processing unit 42 by the communication interface 54. The main processing unit 42 can then process the indicators (via the transmitted data) to determine the relevant respiration rate.

In some embodiments, determined biological parameters can be subjected to statistical analysis. For example, once the main processing unit 42 has determined (or received from the monitoring devices 40) data representing respiration rates of multiple animals, the main processing unit 42 can conduct various statistical analyses on the data in order to extract useful information. In some embodiments, the main processing unit 42 can identify an average respiration rate for a herd in the enclosure 20 (or a subset thereof) based upon averaging determined respiration rates for multiple animals and, as appropriate, can track the average respiration rate over time. In this regard, for example, an appropriate distribution of the monitoring devices 40 through the enclosure 20 may be particularly useful, as it may help to ensure that statistical analysis of the determined biological parameters provides an appropriate representation of the status of the herd as a whole (or a portion thereof).

In some embodiments, other parameters can also be monitored. For example, in addition to recording average respiration rates for a herd over time, the main processing unit 42 can monitor and/or record water consumption by the herd, environmental information for the enclosure 20 (e.g., temperature, humidity, operational status of environment management equipment, and so on), external environmental information (e.g., ambient temperature, humidity and other weather factors outside of the enclosure 20, including actual and predicted temperature, humidity and other weather factors) and so on. As appropriate, these additional parameters can be analyzed in conjunction with the relevant biological parameters (e.g., respiration rates) in order to more effectively control environmental management equipment and/or to glean insights into deployment and calibration of monitoring devices 40, aspects of environmental control (e.g., rates of fan and misting operation), and other aspects of system performance.

Once an appropriate biological parameter (e.g., respiration rate), has been determined, the environmental management equipment can be controlled accordingly. For example, where the main processing unit 42 has determined that the average respiration rate for the herd in the enclosure 20 (or a portion thereof) exceeds a particular threshold, the main processing unit 42 can control the environmental management equipment in order to provide increased cooling to the herd.

Control of the environmental management equipment can be executed in bulk (i.e., with regard to all of the environmental management equipment) or with regard to a subset of the environmental management equipment. For example, depending on the character of the determined biological parameters and/or other factors (e.g., internal or external ambient temperature or humidity, herd water consumption, or other herd behavior, and so on), the main processing unit 42 can selectively activate or deactivate one or more of the fans 36, increase or decrease fan speed for one or more of the fans 36, activate or deactivate part or all of the misting array 38, increase or decrease the misting rate at part or all of the misting array 38, open or close or otherwise move one or more shutters, or various combinations thereof. Likewise, in cold-weather climates, the main processing unit 42 can also (or alternatively) regulate operation of one or more heaters (not shown).

### EXAMPLES

The following Examples are an illustration of one or more examples of embodiments of carrying out the invention and are not intended as to limit the scope of the invention.

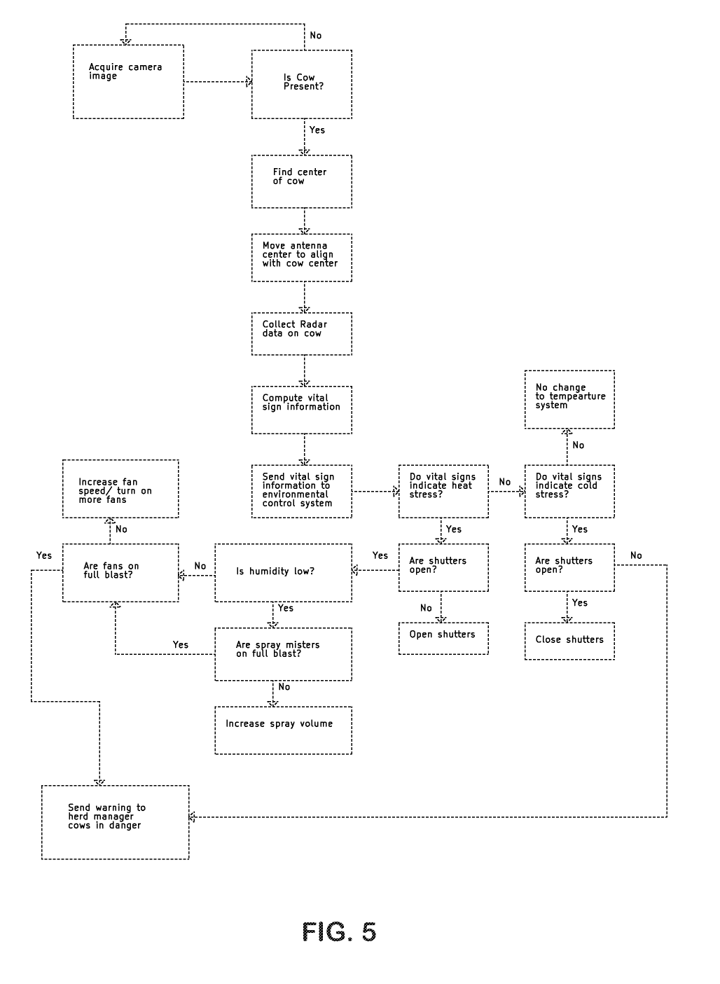

Referring to FIG. 5, a logic diagram illustrating one example of embodiments of operation of the system is shown. In the illustrated example, the system queries whether an animal (e.g., a cow) is present. In this regard, a camera may first acquire an image and provide data relative to that image or the image to the system. If no cow is present the system awaits acquisition of an image of the animal. Sampling of data may be taken at any suitable rate. In one example, sampling may be accomplished every 15 seconds. If a cow is present the system proceeds to locate the approximate center of the cow. The antenna is moved and approximately centered to align with the cow center. Radar data is then collected on the cow. In particular, vital sign data may be collected. The system computes the vital sign information and then sends this information to the environmental control system processor which processes this data. The system queries whether vital signs indicate heat stress. If the answer is no, the system queries whether the vital signs indicate cold stress. If no, there is no change and no change in temperature to the system. If vital signs indicate cold stress, the system queries whether shutters are open. If yes, the system sends a signal to the environmental control system to close the shutters. If no, the system communicates a warning to the herd manager that one or more cows are in danger. If vital signs indicate heat stress, the system queries whether the shutters are open. If the answer is no, the system sends a signal to the environmental control system to open the shutters. If the shutters are open, then the system queries whether humidity is low. If yes, the system queries whether spray misters are on full blast. If no, the system sends a signal to the environmental control system to increase spray volume. If spray misters are on full blast or humidity is not low, then the system queries whether the fans are on full blast. If no, the system sends a signal to the environmental control system to increase fan speed and/or turn on one or more fans. If the fans are on full blast, then a warning is communicated to the herd manager that one or more cows are in danger.

As one example of application of the systems described herein, cow respiration is a direct proxy for heat stress. According to the foregoing embodiments, to combat heat stress in a dairy operation which can be detrimental to cows, a dairy farmer may install one or more cooling systems in a barn that utilizes fans, water misting systems, and the control of shutters to block out the sun. The system disclosed herein is operable to control the temperature in enclosures that contain live animals by measuring the respiration of cows or live animals in the enclosure (in this case the dairy barn) through the use of a radar system. By measuring the average respiration of a group of cows in the barn during the 24 hour day, and using this metric to control the cooling systems, the system ensures that maximum cow comfort is maintained with the minimum of energy consumption.

Some portions of the detailed descriptions herein are presented in terms of procedures, steps, logic blocks, processing, and other symbolic representations of operations on data bits that can be performed on computer memory. These descriptions and representations are the means used by those skilled in the data-processing arts to most effectively convey the substance of their work to others skilled in the art. A procedure, computer-executed step, logic block, process, etc. is here, and generally, conceived to be a self-consistent sequence of steps or instructions leading to a desired result. The steps are those requiring physical manipulations of physical quantities. Usually, though not necessarily, these quantities take the form of electrical or magnetic signals capable of being stored, transferred, combined, compared, and otherwise manipulated in a computer system. It should be borne in mind; however, that all of these and similar terms are to be associated with the appropriate physical quantities and are merely convenient labels applied to these quantities. Unless specifically stated otherwise as apparent from the discussions herein, it is appreciated that throughout the present invention, discussions utilizing terms, such as “receiving,” “sending,” “generating,” “reading,” “invoking,” “selecting,” and the like, refer to the action and processes of a computer system, or similar electronic computing device, including an embedded system, that manipulates and transforms data represented as physical (electronic) quantities within a suitable computer system.

The previous description of the disclosed embodiments is provided to enable any person skilled in the art to make or use the invention. Various modifications to these embodiments will be readily apparent to those skilled in the art, and the generic principles defined herein may be applied to other embodiments without departing from the spirit or scope of the invention. Thus, the invention is not intended to be limited to the embodiments shown herein but is to be accorded the widest scope consistent with the principles and novel features disclosed herein.

The technical effects and technical problems in the specification are exemplary and are not limiting. It should be noted that the embodiments described in the specification may have other technical effects and can solve other technical problems.

In the claims:

1. A system for environmental control of an enclosure for animals, the enclosure including environmental management equipment, the system comprising:
at least one radar device configured to detect an indicator of a biological parameter for at least one of the animals; and
one or more controllers in communication with the radar device and configured to:
determine a value for the biological parameter based upon the detected indicator; and
control operation of the environmental management equipment based upon the determined value for the biological parameter.

2. The system of claim 1, wherein the biological parameter includes a respiration rate.

3. The system of claim 2, wherein the control of the operation of the environmental management equipment includes:
determining an indicator of heat stress for the at least one of the animals based upon the determined value for the respiration rate; and
controlling the environmental management equipment to increase a cooling rate for the animals based upon the determined indicator of heat stress.

4. The system of claim 1, wherein the at least one radar device is configured to detect indicators of the biological parameter for a plurality of the animals; and
wherein the one or more controllers is further configured to:
determine a statistical measure of the biological parameter for the plurality of the animals based upon the detected indicators of the biological parameter; and
control the operation of the environmental management equipment based upon the determined statistical measure.

5. The system of claim 4, wherein the statistical measure of the biological parameter includes an average respiration rate for the plurality of the animals.

6. The system of claim 4, wherein the at least one radar device includes a plurality of radar devices distributed at a plurality of locations in the enclosure.

7. The system of claim 1, wherein the enclosure is an enclosure for a dairy operation and includes an alleyway leading to a milking parlor; and
wherein the at least one radar device is disposed to detect the indicator of the biological parameter for the at least one of the animals as the at least one of the animals moves through the alleyway.

8. The system of claim 7, wherein the at least one radar device is disposed within the alleyway.

9. The system of claim 8, wherein the at least one radar device is included in a plurality of radar devices distributed along the alleyway.

10. The system of claim 1, wherein the enclosure includes at least one watering trough; and
wherein the at least one radar device is disposed to detect the indicator of the biological parameter for the at least one of the animals as the at least one of the animals uses the at least one watering trough.

11. The system of claim 10, wherein the at least one watering trough is included in a plurality of watering troughs; and
wherein the at least one radar device is included in a plurality of radar devices disposed to monitor each watering trough of the plurality of watering troughs.

12. A method for controlling environmental conditions of an enclosure for animals, the enclosure including environmental management equipment, the method comprising:
detecting, with at least one radar device, an indicator of a biological parameter for at least one of the animals
determining, with one or more computing devices, a value for the biological parameter based upon the detected indicator; and
controlling, with the one or more computing devices, operation of the environmental management equipment based upon the determined value for the biological parameter.

13. The system of claim 12, wherein the biological parameter includes a respiration rate.

14. The method of claim 13, wherein controlling the operation of the environmental management equipment includes:
determining an indicator of heat stress for the at least one of the animals based upon the determined value for the respiration rate; and
controlling the environmental management equipment to increase a cooling rate for the animals based upon the determined indicator of heat stress.

15. The method of claim 12, further comprising:
detecting, with the at least one radar device, indicators of the biological parameter for a plurality of the animals;
determining a statistical measure of the biological parameter for the plurality of the animals based upon the detected indicators of the biological parameter; and
controlling the operation of the environmental management equipment based upon the determined statistical measure.

16. The method of claim 15, wherein the statistical measure of the biological parameter includes an average respiration rate for the plurality of the animals.

17. The method of claim 16, wherein the at least one radar device includes a plurality of radar devices distributed at a plurality of locations in the enclosure.

18. The method of claim 12, wherein the enclosure is an enclosure for a dairy operation and includes an alleyway leading to a milking parlor; and
wherein the at least one radar device is disposed to detect the indicator of the biological parameter for the at least one of the animals as the at least one of the animals moves through the alleyway.

19. The method of claim 18, wherein the at least one radar device is disposed within the alleyway.

20. The method of claim 19, wherein the at least one radar device is included in a plurality of radar devices distributed along the alleyway.

21. The method of claim 12, wherein the enclosure includes at least one watering trough; and
wherein the at least one radar device is disposed to detect the indicator of the biological parameter for the at least one of the animals as the at least one of the animals uses the at least one watering trough.

22. The method of claim 21, wherein the at least one watering trough is included in a plurality of watering troughs; and
wherein the at least one radar device is included in a plurality of radar devices disposed to monitor each watering trough of the plurality of watering troughs.

23. A monitoring device for touch-less monitoring of a plurality of animals in an enclosure that includes environmental management equipment, the monitoring device comprising:
at least one radar device configured to detect an indicator of a biological parameter for at least one of the animals;
a communication interface in communication with the environmental management equipment; and
one or more controllers in communication with the radar device and the communication interface; the one or more controllers being configured to:
determine a value for the biological parameter based upon the detected indicator; and
cause the communication interface to communicate control signals to the environmental management equipment based upon the determined value for the biological parameter.

24. The monitoring device of claim 23, wherein the biological parameter includes a respiration rate.

25. The monitoring device of claim 24, wherein the one or more controllers are further configured to determine an indicator of heat stress for the at least one of the animals based upon the determined value for the respiration rate; and
wherein the control signals are configured to cause the environmental management equipment to increase a cooling rate for the animals based upon the determined indicator of heat stress.

26. The monitoring device of claim 23, wherein the at least one radar device is configured to detect indicators of the biological parameter for a plurality of the animals;
wherein the one or more controllers is further configured to determine a statistical measure of the biological parameter for the plurality of the animals based upon the detected indicators of the biological parameter; and
wherein the control signals are determined based upon the determined statistical measure.

27. The monitoring device of claim 26, wherein the statistical measure of the biological parameter includes an average respiration rate for the plurality of the animals.

28. The monitoring device of claim 26, wherein the at least one radar device includes a plurality of radar devices distributed at a plurality of locations in the enclosure.

29. The monitoring device of claim 23, wherein the enclosure is an enclosure for a dairy operation and includes an alleyway leading to a milking parlor; and
wherein the at least one radar device is disposed to detect the indicator of the biological parameter for the at least one of the animals as the at least one of the animals moves through the alleyway.

30. The monitoring device of claim 23, wherein the enclosure includes at least one watering trough; and
wherein the at least one radar device is disposed to detect the indicator of the biological parameter for the at least one of the animals as the at least one of the animals uses the at least one watering trough.
